(19) United States
(12) Patent Application Publication
(10) Pub. No.: US 2015/0342846 A1
(43) Pub. Date: Dec. 3, 2015

(54) Hair Styling Composition

(71) Applicant: Herb UK Limited, Lymington (GB)

(72) Inventors: Raoul John Perfitt, Lymington (GB); Cicely Andrea Ruth Carimbocas, Lymington (GB)

(21) Appl. No.: 14/721,019

(22) Filed: May 26, 2015

(30) Foreign Application Priority Data
May 28, 2014 (GB) 1409432.0

(51) Int. Cl. A61K 8/37; A45D 7/04; A61Q 5/04; A61K 8/22; A61Q 5/06

(52) U.S. Cl. CPC A61K 8/37; A61K 8/22; A61Q 5/06; A61Q 5/04; A45D 7/04

(57) ABSTRACT

The present application relates to a hair styling composition for use in permanently or semi-permanently styling hair, the composition comprising citronellyl methylcrotonate and hydrogen peroxide. The present application also concerns a related hair-styling kit for use in permanently or semi-permanently styling hair as well as associated uses and methods.

The present invention relates to a hair styling composition. In particular, it relates to a hair styling composition comprising citronellyl methylcrotonate.

Hair styling refers to the manipulation of hair into an arrangement which differs from the natural arrangement. This may be done for reasons of fashion, conformity with external requirements (such as an employer's dress code), convenience, or for health reasons.

Techniques for hair styling range from simple physical manipulation, such as combing/brushing; application of an external adhesive, in the form of a hairspray, wax or gel, which are removed by brushing or washing; through to more long-lasting approaches.

For many people, it is desirable to retain a hairstyle for a period of time extending beyond a single day. Thus, techniques have been developed which seek to change the structure of the hair in a longer-lasting manner. In the early 20^{th} Century, this was first achieved using heat treatment. More recently, chemical treatments have been developed in order to alter the shape of hair on a permanent or semi-permanent basis. Such treatments, whether by heat or chemical means, typically seek either to straighten hair, by removing naturally-present curls or waves, or to introduce curls or waves not present naturally (known as a “permanent wave” or “perm”).

Hair contains fibres containing the protein keratin. It is believed that the shape of the hair fibres is maintained, at least in part, by disulfide linkages within the protein. Thus, permanent styling of the hair requires these linkages to be broken so that the hair can be reshaped, followed by the formation of new linkages to maintain the hair in the desired shape. Chemical treatment typically uses a reducing agent to break the linkages, followed by oxidation to form the new linkages.

Heat treatment of hair can leave it brittle and prone to damage, particularly if the treatment is repeated at regular intervals. There is also the risk of burns to the scalp, ears and neck as the heat is applied to the hair, due to the high temperatures involved. Similarly, early chemical treatments involved the use of potent chemicals such as ammonia, with consequent risk of chemical damage or irritation. In particular, the higher levels of risks associated with such treatments make them unsuitable for use outside of professional hair treatment salons.

It is useful, therefore, to have a permanent or semi-permanent hair styling composition which has lower health risks and is suitable for use at home. Examples of such compositions are known, utilising reducing agents such as thioglycolic acid or cysteamine, followed by an oxidising agent such as hydrogen peroxide. One such system is described in EP 2 535 038.

However, the use of such compositions, and particularly those employing thiol-based reducing agents such as cysteamine, can impart an unpleasant odour to the hair, which remains even after neutralisation and subsequent washing. There is therefore a need for an improved hair styling composition in which residual odour is reduced or absent.

According to one aspect of the present invention there is provided a hair styling composition comprising citronellyl methylcrotonate.

As used herein, a hair styling composition is any composition which is added directly to the hair in order to effect hair styling, particularly for use in permanently or semi-permanently styling, such as for example straightening or curling. It will be understood that, although such processes may be achieved using a single composition, typically two or compositions are used sequentially. The composition of the invention may be any one or more of such compositions.

As used herein ‘permanent’ hair styling indicates that the style is not irreversibly lost through processes such as washing, brushing etc. Such distinctions are well understood in the art. Thus, hair which has been permanently curled will still be curly after washing and drying, unless further steps are taken to actively remove the curls. As is well known, however, even ‘permanent’ treatments will not typically affect future hair growth, and so the style will be gradually lost as the treated hair grows out and is replaced.

It has been surprisingly found that citronellyl methylcrotonate is particularly effective at removing any residual odour in the hair resulting from the use of a hair styling composition, and particularly the odour resulting from the use of a thiol-based reducing agent, whilst still allowing the composition to be effective in styling hair.

Preferably, the composition comprises citronellyl methylcrotonate at a level of at least 0.1% by weight. More preferably, the composition comprises at least 0.15%, at least 0.2% at least 0.25%, at least 0.3%, at least 0.35%, at least 0.4%, at least 0.45%, at least 0.5%, at least 0.55%, at least 0.6%, at least 0.65%, at least 0.7% or at least 0.75% citronellyl methylcrotonate. Typically, such levels of methylcrotonate are found in a neutralising component intended for application directly to the hair.

The composition may comprise no more than 2.5% by weight citronellyl methylcrotonate, no more than 2.0%, no more than 1.75%, no more than 1.5%, no more than 1.25%, no more than 1.0%, or no more than 0.75%.

The composition may be in any suitable form. In particular, it may be in the form of a liquid, a gel or a mousse. Preferably, the composition is in the form of a solution in which water is the main solvent by weight, or an emulsion in which water is the main solvent by weight of the continuous phase.

Preferably, the composition is a neutralising component for use with a thiol-containing styling component. Typically, such neutralising components will comprise an oxidising agent capable of re-oxidising the keratin to form new disulfide linkages, and neutralising the reducing effect of the thiol, thereby causing the hair fibres to be re-set in their new arrangement. It has been found that, when added to the hair together with the oxidising agent, the citronellyl methylcrotonate is particularly effective at removing any residual odour resulting from the use of the thiol.

Preferably, the composition further comprises hydrogen peroxide. Hydrogen peroxide is an oxidising agent capable of forming disulfide linkages in the treated hair fibres, and, if used at the appropriate level, is relatively safe for use on the hair and scalp. Preferably, the composition comprises hydrogen peroxide at a level of at least 1.0% by weight. More preferably, the composition comprises at least 1.2% by weight, at least 1.4% by weight, at least 1.6% by weight, at least 1.8% by weight, at least 2.0% by weight, at least 2.2% by weight, at least 2.4% by weight, at least 2.6% by weight, at least 2.8% by weight, at least 2.9% by weight, or at least 2.95% by weight hydrogen peroxide.

The composition may comprise no more than 9% by weight hydrogen peroxide, no more than 8% by weight, no more than 7% by weight, no more than 6% by weight, no more than 5% by weight, no more than 4.5% by weight, no more than 4.0% by weight, no more than 3.5% by weight, or no more than 3.0% by weight hydrogen peroxide.

Preferably, the composition further comprises one or more ingredients which function as surfactants, stabilisers, emulsifiers and/or conditioners. Further preferably, the ingredients comprise one or more amphiphilic compounds. Examples of suitable ingredients include polysorbate 20, sodium C14-16 olefin sulfonate, hydrolysed corn protein, hydrolysed wheat protein, hydrolysed soy protein, and disodium EDTA. Typically, the composition may comprise up to 2% by weight of protein-derived conditioning agents, such as hydrolysed corn protein, hydrolysed wheat protein, and hydrolysed soy protein. More preferably, the composition comprises sodium C14-16 olefin sulfonate. Typically, the composition may comprise at least 0.5% by weight sodium C14-16 olefin sulfonate , at least 0.6% by weight, at least 0.7% by weight, at least 0.8% by weight, at least 0.9% by weight, or at least 1% by weight sodium C14-16 olefin sulfonate. Typically, the composition may comprise no more than 10% by weight sodium C14-16 olefin sulfonate, no more than 9% by weight, no more than 8% by weight, no more than 7% by weight, no more than 6% by weight, no more than 5% by weight, no more than 4% by weight, no more than 3% by weight, no more than 2% by weight, or no more than 1.5% by weight sodium C14-16 olefin sulfonate. Sodium C14-16 olefin sulfonate has been found to be particularly effective as a surfactant in the composition of the present invention.

Furthermore, it avoids the need for sulphate-based surfactants, which can cause irritation of the scalp.

According to a further aspect of the invention, there is provided a hair-styling kit comprising a styling component comprising a thiol, and a neutralising component, wherein the kit comprises citronellyl methylcrotonate.

The kit may further comprise an activating component. If necessary, the activating component is mixed with the styling component. The (activated) styling component is then applied to the hair. The hair may be manipulated into the desired style before, after, or concurrently with application of the styling component. After an appropriate period of time, the neutralising component is applied to the hair. Finally, the hair is rinsed and conditioned.

In some embodiments, the neutralising component is a neutralising component for use with a thiol-containing hair-styling composition, as described above. Thus, the neutralising component may comprise citronellyl methylcrotonate in addition to, for example, an oxidising agent.

Alternatively, the kit may comprise a styling component comprising a thiol, a neutralising component, and a further component comprising citronellyl methylcrotonate. In such embodiments, the further component comprising citronellyl methylcrotonate may be added to the neutralising component shortly before application to the hair. The kit may comprise sufficient citronellyl methylcrotonate such that, when the further component is added to the neutralising component, the neutralising agent comprises citronellyl methylcrotonate in a proportion as set out above.

Preferably, the kit comprises a styling component comprising at least 5% by weight, at least 7.5% by weight, at least 10%, at least 12.5% or at least 15% by weight thiol. The styling component may comprise no more than 20.0% by weight, no more than 18% by weight, no more than 17% by weight, no more than 16% by weight, or no more than 15.0% by weight thiol.

Preferably, the thiol comprises cysteamine or a salt thereof. More preferably, the thiol comprises cysteamine hydrochloride.

Preferably, the styling component further comprises one or more ingredients which function as stabilisers, emulsifiers and/or conditioners. Further preferably, the ingredients comprise one or more amphiphilic compounds. Examples of suitable ingredients include polysorbate 20, oleth-10, PVP, amodimethicone, cetrimonium chloride, trideceth-12 and disodium EDTA.

Preferably, the kit further comprises an activating component for activating the styling component. Preferably, the activating component comprises a base, more preferably an organic base. Preferably, the activating component comprises triethanolamine. Preferably, the activating component further comprises a thickener, such as for example glycerine. Preferably the kit is substantially free from ammonia.

According to a further aspect of the invention there is provided the use of citronellyl methylcrotonate to reduce the residual odour associated with use of a thiol-containing hair styling composition.

According to a further aspect of the invention there is provided a method of styling hair, comprising treating the hair with a styling component comprising cysteamine, and subsequently treating the hair with a neutralising component comprising citronellyl methylcrotonate.

Preferably, the styling component and/or neutralising component are as set out above.

The following are further aspects of the present invention:

(1) A hair styling composition comprising citronellyl methylcrotonate.

(2) The composition as defined in (1), which comprises at least 0.1% by weight citronellyl methylcrotonate.

(3) The composition as defined in (1) or (2), wherein the composition is a neutralising component for use with a thiol-containing hair-styling composition.

(4) The neutralising component as defined in (3), further comprising hydrogen peroxide.

(5) The neutralising component as defined in (4), which comprises at least 1.0% by weight hydrogen peroxide.

(6) The composition as defined in any one of (1) to (5), further comprising sodium C14-16 olefin sulfonate.

(7) A hair-styling kit comprising a styling component comprising a thiol and a neutralising component, wherein the kit comprises citronellyl methylcrotonate.

(8) A hair styling kit as defined in (7), wherein the neutralising component is as defined in any one of (3) to (6).

(9) The kit as defined in (7) or (8), wherein the thiol comprises cysteamine

(10) The kit as defined in any one of (7) to (9), wherein the styling component comprises at least 5% by weight cysteamine

(11) The kit as defined in any one of (7) to (10), further comprising an activating component comprising a base.

(12) The kit as defined in (11), wherein the base comprises triethanolamine

(13) The kit as defined in any one of (7) to (12), wherein the kit is substantially free from ammonia.

(14) Use of citronellyl methylcrotonate to reduce the residual odour associated with use of a thiol-containing hair styling composition.

(15) A method of styling hair, comprising treating the hair with a styling component comprising cysteamine, and subsequently treating the hair with a neutralising component comprising citronellyl methylcrotonate.

Aspects of the invention are illustrated by the following specific Example.

A styling component has the following formulation:

| Ingredient | wt % |
|---|---|
| Aqua | 80.135 |
| Cysteamine hydrochloride | 15.000 |
| Polysorbate 20 | 2.500 |
| Oleth-10 | 1.000 |
| Parfum | 0.500 |
| PVP | 0.500 |
| Amodimethicone | 0.175 |
| Cetrimonium chloride | 0.010 |
| Tridceth-12 | 0.010 |
| Disodium EDTA | 0.150 |
| BHA | 0.020 |

An activating component has the following formulation:

| Ingredient | wt % |
|---|---|
| Aqua | 68.000 |
| Triethanolamine | 30.000 |
| Glycerine | 2.000 |

A neutralising component has the following formulation:

| Ingredient | wt % |
|---|---|
| Aqua | 90.032 |
| Hydrogen peroxide | 2.975 |
| Polysorbate 20 | 2.000 |
| Sodium C14-16 olefin sulfonate | 1.233 |
| Hydrolysed corn protein | 0.780 |
| Citric acid | 0.750 |
| Hydrolysed wheat protein | 0.640 |
| Hydrolysed soy protein | 0.540 |
| Anthemis Nobilis (chamomile) | 0.500 |
| flower extract | |
| Parfum | 0.400 |
| Disodium EDTA | 0.100 |
| Salicylic acid | 0.050 |

to which 0.75 wt % citronellyl methylcrotonate is added immediately before use.

In use, the hair is held in the desired style using rollers/rods. 70 ml of the styling component is mixed with the required amount of activating component to raise the pH as necessary according to hair type (e.g. 30 ml for virgin and resistant grey coarse hair, 15 to 20 ml for coloured hair, no activator for bleached or heavily highlighted hair). The resulting mixture is applied to the hair, and left in place for a suitable length of time dependent on the condition of the hair (typically, up to 20 mins for virgin hair, but potentially up to a maximum of 30 mins), and is then rinsed off using water (as hot as possible) for 10 to 15 mins.

The citronellyl methylcrotonate is added to the neutralising component, which is then applied to the hair. Three-quarters of the neutraliser is applied first, with the perm rods in place, and left for 5 mins. The rods are then removed and the remaining neutraliser applied, massaged into the hair, and left for a further 3 mins. The neutralising component is then rinsed out with warm water, and the hair is treated with a suitable hair conditioner. Testing was carried out on hair swatches over a 6 week period, both to assess the durability of the styling, and any residual smell. Swatches were styled by curling round a cylinder and then hung. Some slight curl drop was observed, but this was comparable with that obtained using alternative styling formulations. When compared against swatches treated with a similar formulation lacking citronellyl methylcrotonate, the present formulation was consistently rated by a 10-person panel as resulting in hair having a more pleasant odour.

1. A hair styling composition for use in permanently or semi-permanently styling hair, the composition comprising citronellyl methylcrotonate and hydrogen peroxide.

2. The composition as claimed in claim 1, which comprises at least 0.1% by weight citronellyl methylcrotonate.

3. The composition as claimed in claim 1, wherein the composition is a neutralising component for use with a thiol-containing hair-styling composition.

4. The composition as claimed in claim 1, which comprises at least 1.0% by weight hydrogen peroxide.

5. The composition as claimed in claim 1, further comprising sodium C14-16 olefin sulfonate.

6. A hair-styling kit for use in permanently or semi-permanently styling hair, the kit comprising: (i) a styling component comprising a thiol and (ii) a neutralising component comprising hydrogen peroxide; wherein the kit comprises citronellyl methylcrotonate.

7. The kit as claimed in claim 6, wherein the neutralising component comprises citronellyl methylcrotonate.

8. The kit as claimed in claim 6, wherein the thiol comprises cysteamine.

9. The kit as claimed in claim 6, wherein the styling component comprises at least 5% by weight cysteamine.

10. The kit as claimed in claim 6, further comprising an activating component comprising a base.

11. The kit as claimed in claim 10, wherein the base comprises triethanolamine.

12. The kit as claimed in claim 6, wherein the kit is substantially free from ammonia.

13. A method of permanently or semi-permanently styling hair, comprising treating the hair with a styling component comprising cysteamine, and subsequently treating the hair with a neutralising component comprising citronellyl methylcrotonate and hydrogen peroxide.
